(19) United States
(12) Patent Application Publication
(10) Pub. No.: US 2026/0308672 A1
(43) Pub. Date: Oct. 8, 2026

(54) Method and System for Improving Specificity of Analyte Detection Using Real-Time Nucleic Acid Amplification

(71) Applicant: Gen-Probe Incorporated, San Diego, CA (US)

(72) Inventors: Vincent Chabot, Escondido, CA (US); David Opalsky, San Diego, CA (US)

(73) Assignee: Gen-Probe Incorporated, San Diego, CA (US)

(21) Appl. No.: 19/481,005

(22) PCT Filed: May 3, 2024
(86) PCT No.: PCT/US2024/027803

§ 371 Date: Nov. 3, 2025

Related U.S. Application Data
(60) Provisional application No. 63/500,475, filed on May 5, 2023.

(51) Int. Cl. C12Q 1/6851; G16B 20/10

(52) U.S. Cl. CPC C12Q 1/6851; G16B 20/10

(57) ABSTRACT

Methods and systems including programmed computers that can be used for improving nucleic acid analyte detection using real-time amplification and monitoring, where closely related nucleic acid sequences can be resolved from each other. In one embodiment, detection of target sequences differing from each other by a single nucleotide were resolved by applying a mathematical transformation. In another embodiment, two target sequences differing from each other by a single nucleotide were detected and resolved from each other using a labeled probe specific for only one of the two target sequences.

### RELATED APPLICATION

This application claims the benefit of U.S. Provisional Application No. 63/500,475, filed May 5, 2023. The entire disclosure of this related application is hereby incorporated by reference.

### FIELD

The present disclosure relates generally to the field of nucleic acid detection. More specifically, the disclosure relates to methods, systems, and software useful for detecting a target nucleic acid with reduced false-positive errors.

### BACKGROUND

Molecular diagnostic testing relies heavily on detection of nucleic acid sequences with high specificity. Many different laboratory techniques, including allele-specific nucleic acid amplification, sequence-specific hybridization probe binding, signal amplification, and nucleic acid sequencing have been used for this purpose. Target sequences that may be of interest include single-base sequence variants, insertions of one or more nucleotides, or deletion of one or more nucleotides.

One category of target sequences that may be of particular interest in certain clinical applications involves very subtle base changes from a conventional or wild-type starting sequence. In one example, KRAS is a well-known oncogene that is frequently mutated at characteristic nucleotide positions in a variety of cancers (Forrester et al., Nature 327:298-303 (1987)). These cancers include pancreatic ductal adenocarcinomas, colorectal adenocarcinomas, and lung adenocarcinomas. Most oncogenic KRAS mutations occur at codons 12, 13, and 61. Using KRAS codon 12 as an example, at least four different point mutations in this single codon have been identified in humans (e.g., Gao et al., Theranostics 10:5137-5153 (2020)). Other examples of point mutations having biological impacts relate to drug resistant phenotypes. Falling under this category would be drug-resistant Mycobacterium tuberculosis (see Honoré et al., Antimicrobial Agents and Chemotherapy 38:238-242 (1994)) and drug-resistant Mycoplasma genitalium (see WO 2022/016153 A1).

To overcome difficulty in obtaining sufficient sample material for analysis, the process of detecting and/or quantifying target nucleic acids frequently involves a preliminary step for synthesizing nucleic acid amplification products. More than one amplification product frequently is synthesized in an amplification reaction, but detection of only one nucleic acid amplification product may be desired. For example, a wild-type sequence and a single nucleotide polymorphism (SNP) may be amplified in the same reaction mixture, but only the amplification product containing the SNP is of interest. Difficulties frequently arise when detection of a non-target (e.g., wild-type) nucleic acid leads to the false conclusion that a target nucleic acid (e.g., SNP) is present in a test sample. Such instances are referred to a “false-positive” results.

The present disclosure details one approach that can be used to reduce the incidence of false-positive results arising from detection signals produced by hybridization of sequence-specific probes to non-target nucleic acid amplification products. Advantageously, the approach avoids the need for re-designing the assay chemistry by substituting modified amplification and detection oligonucleotides.

### SUMMARY

Provided herein are the following embodiments.

Embodiment 1 is a method of determining whether a test sample suspected of including nucleic acid templates for a nucleic acid amplification reaction includes a target nucleic acid, the method including the steps of: (a) acquiring or having acquired a real-time run curve data set including signal data representing production of an amplification product in the nucleic acid amplification reaction as a function of a reaction progress parameter, wherein the nucleic acid amplification reaction uses any of the target nucleic acid and a non-target nucleic acid that may have been present in the test sample as templates to produce the amplification product; (b) calculating or having calculated a first derivative of the real-time run curve data set, including magnitude values of the first derivative; (c) comparing or having compared the calculated magnitude values of the first derivative with a first threshold value; and (d) determining or having determined either that (i) the test sample includes the target nucleic acid if any calculated magnitude value of the first derivative met or exceeded the first threshold value, or (ii) the test sample does not include the target nucleic acid if no calculated magnitude value of the first derivative met or exceeded the first threshold value.

Embodiment 2 is the method of embodiment 1, wherein the real-time run curve data set includes fluorescence magnitude readings, and wherein the method further includes: comparing the real-time run curve data set with a fluorescence threshold value and determining that at least one data point in the real-time run curve data set has a fluorescence magnitude that exceeds the fluorescence threshold value, and determining that the test sample includes the non-target nucleic acid if it is determined in step (d) that the test sample does not include the target nucleic acid.

Embodiment 3 is the method of either embodiment 1 or embodiment 2, wherein the target nucleic acid and the non-target nucleic acid differ from each other at only a single nucleotide position.

Embodiment 4 is the method of any one of embodiments 1 to 3, wherein the test sample includes the target nucleic acid, and wherein the method further includes a step of quantifying or having quantified the target nucleic acid present in the test sample.

Embodiment 5 is the method of embodiment 4, wherein the step of quantifying or having quantified includes first determining a maximum value of the first derivative from step (b), and then using the maximum value of the first derivative together with the reaction progress parameter as an indicator of the amount of the target nucleic acid present in the test sample.

Embodiment 6 is the method of any one of embodiments 1 to 5, further including a step of preparing a non-transient record of the result from step (d).

Embodiment 7 is the method of embodiment 6, wherein the non-transient record includes printing on paper, or recording on computer-readable storage media.

Embodiment 8 is the method of any one of embodiments 4 to 7, further including a step of preparing a non-transient record of the result from the step of quantifying or having quantified.

Embodiment 9 is the method of embodiment 8, wherein the non-transient record includes printing on paper, or recording on computer-readable storage media.

Embodiment 10 is the method of any one of embodiments 1 to 9, wherein the reaction progress parameter of step (a) is measured in cycle numbers, wherein the nucleic acid amplification reaction includes a PCR reaction, and wherein the first threshold value is a predetermined threshold value.

Embodiment 11 is the method of any one of embodiments 1 to 9, wherein the reaction progress parameter of step (a) is either a measure of reaction time or a measure of reaction cycle number.

Embodiment 12 is the method of any one of embodiments 1 to 11, wherein step (a) includes performing the nucleic acid amplification reaction and monitoring synthesis of amplification products as the nucleic acid amplification reaction is occurring.

Embodiment 13 is the method of any one of embodiments 1 to 11, wherein step (a) includes receiving the real-time run curve data set as a computer-readable data file.

Embodiment 14 is the method of any one of embodiments 1 to 13, wherein before step (b) the real-time run curve data set acquired in step (a) is processed using at least one of (i) baseline subtraction, (ii) curve normalization using a curve parameter, and (iii) curve fitting.

Embodiment 15 is the method of embodiment 14, wherein the real-time run curve data set acquired in step (a) is processed using curve fitting, and wherein the curve fitting includes optimizing coefficients of an equation to result in an optimized equation.

Embodiment 16 is the method of any one of embodiments 1 to 15, wherein the real-time run curve data set includes fluorescent readings measured as a function of the reaction progress parameter, and wherein the reaction progress parameter is measured in reaction cycles.

Embodiment 17 is the method of any one of embodiments 1 to 16, wherein step (b) includes calculating with a computer, and wherein step (c) includes comparing with the computer.

Embodiment 18 is the method of any one of embodiments 1 to 17, wherein the nucleic acid amplification reaction is performed using an automated nucleic acid analyzer configured to isolate nucleic acid from the test sample and then perform the nucleic acid amplification reaction using the isolated nucleic acid, and wherein step (b) includes calculating with a computer in communication with the automated nucleic acid analyzer, and wherein step (c) includes comparing with the computer.

Embodiment 19 is the method of any one of embodiments 1 to 18, wherein the first threshold value is a numerical constant.

Embodiment 20 is the method of any one of embodiments 1 to 19, wherein the target nucleic acid is a target nucleic acid isolated from a human pathogen.

Embodiment 21 is the method of embodiment 20, wherein the human pathogen is either a bacterial pathogen or a viral pathogen.

Embodiment 22 is a computer programmed with software instructions to determine whether a target nucleic acid is included in a test sample, the software instructions, when executed by the computer, cause the computer to: (a) receive a real-time run curve data set including signal data that indicates amplification of the target nucleic acid and a non-target nucleic acid in a nucleic acid amplification reaction as a function of a reaction progress parameter, (b) calculate a first derivative of the real-time run curve data set or a processed version thereof, including magnitude values of the first derivative; (c) compare the calculated magnitude values of the first derivative with a first threshold value; and (d) determine either that (i) the test sample included the target nucleic acid if any calculated magnitude value of the first derivative met or exceeded the first threshold value, or (ii) the test sample did not include the target nucleic acid if no calculated magnitude value of the first derivative met or exceeded the first threshold value.

Embodiment 23 is the computer of embodiment 22, wherein the software instructions, when executed by the computer, further cause the computer to compare the real-time run curve data set with a fluorescence threshold value to determine whether any signal data of the real-time run curve data set has a magnitude that meets or exceeds the fluorescence threshold value, and determine that the test sample includes the non-target nucleic acid that differs from the target nucleic acid if the computer determines that the magnitude meets or exceeds the fluorescence threshold value and if the computer determines in (d) that the test sample did not include the target nucleic acid.

Embodiment 24 is the computer of either embodiment 22 or embodiment 23, wherein the signal data in (a) that indicates amplification of the target nucleic acid and the non-target nucleic acid includes fluorescent signal data.

Embodiment 25 is the computer of any one of embodiments 22 to 24, wherein the software instructions, when executed by the computer, further cause the computer to (e) generate a non-transient record of the result from (d).

Embodiment 26 is the computer of any one of embodiments 22 to 25, wherein the software instructions, when executed by the computer, cause the computer to prepare the processed version of the real-time run curve data set, and then (b) calculate the first derivative of the processed version the real-time run curve data set.

Embodiment 27 is the computer of any one of embodiments 22 to 25, wherein the software instructions, when executed by the computer, further cause the computer to prepare the processed version of the real-time run curve data set by performing at least one of (i) baseline subtraction, (ii) curve normalization using a curve parameter, and (iii) curve-fitting; and wherein (b) includes calculate the first derivative of the processed version of the real-time run curve data set.

Embodiment 28 is the computer of any one of embodiments 22 to 27, wherein the first threshold value in (c) is a numerical constant.

Embodiment 29 is the computer of any one of embodiments 22 to 28, wherein the non-transient record in (e) is stored electronically on a computer hard drive.

Embodiment 30 is the computer of any one of embodiments 22 to 29, wherein the computer is in communication with a thermal cycling device equipped with a fluorometer.

Embodiment 31 is a system that determines whether a target nucleic acid is included in a test sample, the system including: a nucleic acid analyzer configured to amplify the target nucleic acid and a non-target nucleic acid in a nucleic acid amplification reaction, wherein the nucleic acid amplification reaction uses any of the target nucleic acid and the non-target nucleic acid that may have been present in the test sample as templates to produce an amplification product, and wherein the nucleic acid analyzer monitors synthesis of the amplification product in the nucleic acid amplification reaction as a function of a reaction progress parameter, whereby there is produced a real-time run curve data set including signal data as a function of the reaction progress parameter, and a computer in communication with the nucleic acid analyzer, the computer being programmed with a set of software instructions causing the computer to (a) calculate a first derivative of the real-time run curve data set or a processed version thereof, including magnitude values of the first derivative; (b) compare the calculated magnitude values of the first derivative with a first threshold value; (c) determine either that (i) the test sample included the target nucleic acid if any calculated magnitude value of the first derivative met or exceeded the first threshold value, or (ii) the test sample did not include the target nucleic acid if no calculated magnitude value of the first derivative met or exceeded the first threshold value; and (d) generate a non-transient record of the result from (c).

Embodiment 32 is the system of embodiment 31, wherein the set of software instructions further cause the computer to compare the real-time run curve data set with a fluorescence threshold value to determine whether any signal data of the real-time run curve data set has a magnitude that meets or exceeds the fluorescence threshold value, and determine that the test sample includes the non-target nucleic acid that differs from the target nucleic acid if the computer determines that the magnitude meets or exceeds the fluorescence threshold value and if the computer determines in (d) that the test sample did not include the target nucleic acid.

Embodiment 33 is the system of either embodiment 31 or embodiment 32, wherein the set of software instructions further cause the computer to calculate a quantity of the target nucleic acid included in the test sample.

Embodiment 34 is the system of any one of embodiments 31 to 33, wherein the set of software instructions further cause the computer to prepare the processed version of the real-time run curve data set by performing at least one of (i) baseline subtraction, (ii) curve normalization using a curve parameter, and (iii) curve-fitting, and wherein (a) includes calculate the first derivative of the processed version of the real-time run curve data set.

Embodiment 35 is the system of any one of embodiments 31 to 34, wherein the computer is a stand-alone computer that is not physically joined to the nucleic acid analyzer.

Embodiment 36 is the system of any one of embodiments 31 to 35, wherein the computer is in communication with an electronic storage device, and wherein the electronic storage device stores an electronic form of the non-transient record generated by the computer.

Embodiment 37 is the system of any one of embodiments 31 to 36, wherein the computer is in communication with a printer that produces the non-transient record.

Embodiment 38 is the system of any one of embodiments 31 to 37, wherein the nucleic acid analyzer includes a fluorometer that detects fluorescent signals produced in the nucleic acid amplification reaction, and wherein the fluorometer is used to monitor synthesis of the amplification product in the nucleic acid amplification reaction.

### BRIEF DESCRIPTION OF THE DRAWINGS

FIG. 1 schematically illustrates a nucleic acid target strand (thick horizontal rectangle) that was amplified using a pair of oppositely disposed (forward and reverse) primers. A single amplification reaction included only one nucleic acid target that may have been either a wild-type target, or one of five SNP-containing nucleic acid targets, where the SNP was located in the filled region underlying the set of hydrolysis probes (narrow horizontal rectangles; “x” indicating single nucleotide differences between probes). Reactions also included all five SNP-specific probes, where only one probe of the illustrated set would have been fully complementary to a SNP-containing amplification product, and where none of the illustrated SNP probes would have been fully complementary to the amplified wild-type sequence. Fluorophores and quenchers, which were the same for all illustrated SNP probes, have been omitted from the schematic. Wild-type probe harboring a distinguishable fluorophore/quencher combination also has been omitted.

FIGS. 2A and 2B present graphical results obtained from nucleic acid amplification reactions using as templates either SNP-containing nucleic acid targets (thin lines), wild-type nucleic acid targets (heavy lines), or negative control reactions (dashed lines) that omitted template nucleic acids. FIG. 2A is a real-time run curve plot of fluorescence (y-axis) as a function of reaction cycle number (x-axis). A horizontal threshold line drawn at 1,000 RFU (relative fluorescence units) was used for determining Ct values. Results from the negative control uniformly appear below the threshold. Amplified nucleic acids were detected using SNP-specific hydrolysis probes that included FAM-labels. FIG. 2B is a first derivative plot of results appearing in FIG. 2A, showing RFU/cycle (y-axis) plotted as a function of reaction cycle number (x-axis). Only curves resulting from the first derivative of run curves obtained using SNP-containing variant nucleic acid targets exceeded the horizontal threshold line drawn at 500 RFU.

FIGS. 3A and 3B are perspective views of an automated nucleic acid analyzer.

### DEFINITIONS

The following terms have the indicated meanings in the specification unless expressly indicated to have a different meaning.

The terms “a,” “an,” and “the” include plural referents, unless the context clearly indicates otherwise. For example, “a nucleic acid” as used herein is understood to represent one or more nucleic acids. As such, the terms “a” (or “an”), “one or more,” and “at least one” can be used interchangeably herein.

A “polynucleotide” is a polymeric form of nucleotides, including ribonucleotides and/or deoxyribonucleotides, of any length. This term refers only to the primary structure of the molecule. Thus, this term embraces double- and single-stranded DNA and RNA (e.g., nucleic acids). It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

As used herein, a “test sample” is any sample to be investigated for the presence of a particular polynucleotide sequence. Test samples include any nucleic acid-containing material obtained from a human, animal, environmental, or laboratory-derived or synthetic sample. Preferred test samples include bodily fluid samples. Whole blood, plasma, and serum are particularly preferred examples of test samples. Other test samples include swab samples (e.g., oral, nasal, throat, or vaginal swab samples) saliva, urine, etc.

As used herein, an “analyte” is a chemical or biochemical species that is to be detected and/or quantified. For example, a “polynucleotide analyte” refers to a polynucleotide (e.g., a segment of a viral nucleic acid, or of a bacterial ribosomal nucleic acid) that is to be detected or quantified in a test procedure.

As used herein, a “nucleic acid analyzer” is an apparatus that amplifies, detects, and optionally quantifies nucleic acid analytes. Certain preferred nucleic acid analyzers include a temperature-controlled incubator (e.g., a block, plate, or chamber), a fluorometer in optical communication with contents of the temperature-controlled incubator, and one or more computers or processors that process data gathered by the fluorometer to quantify a nucleic acid analyte of interest.

An “amplification product” (sometimes “amplicon”) is a polynucleotide product of an amplification reaction, wherein a target polynucleotide sequence of a polynucleotide analyte served as the template for synthesis of polynucleotide copies or amplification products. Preferred amplification products include or comprise DNA.

The term “amplify” is used in the broad sense to mean creating an amplification product that can be synthesized enzymatically with a DNA or RNA polymerase (including a reverse transcriptase). By “amplification” or “nucleic acid amplification” or “polynucleotide amplification” and the like is meant any known procedure for obtaining multiple copies, allowing for RNA and DNA equivalents, of a target polynucleotide sequence or its complement or fragments thereof. “Multiple copies” mean at least two copies. A “copy” does not necessarily mean perfect sequence complementarity or identity to the template sequence. Methods for amplifying mRNA are generally known in the art, and include reverse transcription PCR (RT-PCR). Another method which may be used is quantitative PCR (or Q-PCR).

As used herein, the terms “coamplify” and “coamplifying” and variants thereof refer to a process wherein different target polynucleotide sequences are amplified in a single (i.e., the same) amplification reaction. For example, a nucleic acid analyte and an unrelated internal calibrator nucleic acid are “coamplified” when both nucleic acids are amplified in reactions taking place in a single tube, and when both amplification reactions share at least one reagent (e.g., deoxyribonucleotide triphosphates, enzyme, primer(s), etc.) in common.

As used herein, “thermal cycling” refers to repeated changes of temperature, (i.e., increases or decreases of temperature) in a reaction mixture. Samples undergoing thermal cycling may shift from one temperature to another, stabilize at that temperature, transition to a second temperature or return to the starting temperature. The temperature cycle may be repeated as many times as required to study or complete the particular chemical reaction of interest.

By “target” or “target nucleic acid” is meant a nucleic acid containing a sequence that is to be amplified, detected and/or quantified. A target nucleic acid sequence that is to be amplified preferably will be positioned between two oppositely disposed oligonucleotides, and will include the portion of the target nucleic acid that is complementary to each of the oligonucleotides.

By “target nucleic acid sequence” or “target sequence” or “target region” is meant a specific deoxyribonucleotide or ribonucleotide sequence comprising all or part of the nucleotide sequence of a single-stranded target nucleic acid molecule, and the deoxyribonucleotide or ribonucleotide sequence complementary thereto.

By “transcription-associated amplification” is meant any type of polynucleotide amplification that uses an RNA polymerase to produce multiple RNA transcripts from a polynucleotide template. Conventionally, these amplification reactions employ at least one primer having a 3′-end that can be extended by the activity of a DNA polymerase. One example of a transcription-associated amplification method, called “Transcription Mediated Amplification” (TMA), generally employs an RNA polymerase, a DNA polymerase, deoxyribonucleoside triphosphates, ribonucleoside triphosphates, and a promoter-containing oligonucleotide complementary to the target polynucleotide. Variations of TMA are well known in the art as disclosed in detail in Burg et al., U.S. Pat. No. 5,437,990; Kacian et al., U.S. Pat. Nos. 5,399,491 and 5,554,516; Kacian et al., PCT No. WO 93/22461; Gingeras et al, PCT No. WO 88/01302; Gingeras et al., PCT No. WO 88/10315; Malek et al., U.S. Pat. No. 5,130,238; Urdea et al., U.S. Pat. Nos. 4,868,105 and 5,124,246; McDonough et al., PCT No. WO 94/03472; and Ryder et al., PCT No. WO 95/03430. Other transcription-associated amplification methods employing only a single primer that can be extended by a DNA polymerase, as disclosed in the U.S. Pat. No. 7,374,885 are particularly embraced by the definition and are highly preferred for use in connection with the method disclosed herein.

As used herein, an “oligonucleotide” or “oligomer” or “oligo” is a polymeric chain of at least two, generally between about five and about 100, chemical subunits, each subunit comprising a nucleotide base moiety, a sugar moiety, and a linking moiety that joins the subunits in a linear spatial configuration. Common nucleotide base moieties are guanine (G), adenine (A), cytosine (C), thymine (T) and uracil (U), although other rare or modified nucleotide bases able to hydrogen bond are well known to those skilled in the art. Oligonucleotides may optionally include analogs of any of the sugar moieties, the base moieties, and the backbone constituents. Preferred oligonucleotides of the present disclosure fall in a size range of about 10 to about 100 residues. Oligonucleotides may be purified from naturally occurring sources, but preferably are synthesized using any of a variety of well-known enzymatic or chemical methods.

By “amplification oligonucleotide” or “amplification oligomer” is meant an oligomer that hybridizes to a target polynucleotide, or its complement, and participates in a nucleic acid amplification reaction. Examples of amplification oligomers include primers that contain a 3′-end that is extended as part of the amplification process, but also include oligomers that are not extended by a polymerase (e.g., a 3′-blocked oligomer) but may participate in, or facilitate efficient amplification from a primer. Preferred size ranges for amplification oligomers include those that are about 10 to about 80 nucleotides long, or 10 to about 60 nucleotides long and contain at least about 10 contiguous bases, and more preferably at least 12 contiguous bases that are complementary to a region of the target polynucleotide sequence (or a complementary strand thereof). The contiguous bases are preferably at least about 80%, more preferably at least about 90%, and most preferably about 100% complementary to the target sequence to which an amplification oligomer binds. An amplification oligomer may optionally include modified nucleotides or analogs, or additional nucleotides that participate in an amplification reaction but are not complementary to or contained in the target polynucleotide. An amplification oligomer that is 3′-blocked but capable of hybridizing to a target polynucleotide and providing an upstream promoter sequence that serves to initiate transcription is referred to as a “promoter provider” oligomer.

A “primer” is an amplification oligomer that hybridizes to a target polynucleotide template and has a 3′-OH end that can be extended by a DNA polymerase. The 5′ region of the primer may be non-complementary to the target polynucleotide (e.g., a non-complementary promoter sequence), resulting in an oligomer referred to as a “promoter-primer.” Those skilled in the art will appreciate that any oligomer that can function as a primer can be modified to include a 5′ promoter sequence, and thus could function as a promoter-primer. Similarly, any promoter-primer can be modified by removal of, or synthesis without, a promoter sequence and still function as a primer.

As used herein, a “probe” is an oligonucleotide that hybridizes specifically to a target sequence in a polynucleotide, preferably in an amplified polynucleotide, under conditions that promote hybridization, to form a detectable hybrid. Certain preferred probes include a detectable label (e.g., a fluorescent label or chemiluminescent label). Hydrolysis probes conventionally include a fluorescent label and a quencher moiety.

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical, or chemical means.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

As used herein, a “nucleic acid analyzer” is an apparatus or instrument that amplifies, detects, and optionally quantifies nucleic acid analytes. Certain preferred nucleic acid analyzers include a temperature-controlled incubator (e.g., a block, plate, or chamber), a fluorometer in optical communication with contents of the temperature-controlled incubator, and one or more computers or processors that process data gathered by the fluorometer to quantify a nucleic acid analyte of interest. In some embodiments, preferred nucleic acid analyzers perform enzyme-based reactions that amplify or increase the number of copies of a target nucleic acid that is to be quantified. In other embodiments, “signal amplification” is used to detect and/or quantify the target nucleic acid that is to be quantified. An example signal amplification system is provided by the “serial invasive signal amplification reaction” disclosed by Hall et al., in Proc. Natl. Acad. Sci. USA 97:8272-8277 (2000).

As used herein, “time-dependent” monitoring of polynucleotide amplification, or monitoring of polynucleotide amplification in “real-time” refers to a process wherein the amount of amplicon present in an amplification reaction is measured as a function of reaction time or cycle number, and then used to determine a starting amount of template that was present in the reaction mixture at the time the amplification reaction was initiated. For example, the amount of amplicon can be measured prior to commencing each complete cycle of an amplification reaction that comprises thermal cycling, such as PCR. Alternatively, isothermal amplification reactions that do not require physical intervention to initiate the transitions between amplification cycles can be monitored continuously, or at regular time intervals to obtain information regarding the amount of amplicon present as a function of time.

As used herein, a “run curve” (sometimes “growth curve” herein) refers to the characteristic pattern of appearance of a synthetic product, such as an amplicon, in a reaction as a function of time or cycle number (i.e., reaction progress parameters). A run curve is conveniently represented as a two-dimensional plot of time or cycle number (x-axis) against some indicator of product amount, such as a fluorescence measurement (y-axis). Some, but not all, run curves have a sigmoid-shape.

As used herein, the “baseline phase” of a growth curve refers to the initial phase of the curve wherein the amount of product (such as an amplicon) increases at a substantially constant rate, this rate being less than the rate of increase characteristic of the growth phase (which may have a log-linear profile) of the growth curve. The baseline phase of a growth curve typically has a very shallow slope, frequently approximating zero.

As used herein, the “growth phase” of a growth curve refers to the portion of the curve wherein the measurable product substantially increases with time. Transition from the baseline phase into the growth phase in a typical polynucleotide amplification reaction is characterized by the appearance of amplicon at a rate that increases with time. Transition from the growth phase to the plateau phase of the growth curve begins at an inflection point where the rate of amplicon appearance begins to decrease.

As used herein, the “plateau phase” of a triphasic growth curve refers to the final phase of the curve. In the plateau phase, the rate of measurable product formation generally is substantially lower than the rate of amplicon production in the log-linear phase, and may even approach zero.

As used herein, the phrase “indicia of amplification” refers to features of real-time run curves which indicate a predetermined level of progress in polynucleotide amplification reactions. In some embodiments, the time or cycle number at which a threshold level of fluorescence is achieved serves as the indicia of amplification. Such indicia are commonly determined by mathematical analysis of run curves, sometimes referred to as “growth curves,” which display a measurable signal (such as a fluorescence reading) whose intensity is related to the quantity of an amplicon present in a reaction mixture as a function of time, cycle number, etc.

As used herein, the phrase “threshold-based indicia of amplification” refers to indicia of amplification that measure the time or cycle number when a growth curve signal crosses an arbitrary value or threshold (e.g., a threshold fluorescence value). Cycle threshold (Ct) and TTime values are examples of threshold-based indicia of amplification, while TArc and OTArc determinations are examples of non-threshold-based indicia of amplification.

As used herein, the phrase “time-dependent indicia of amplification” refers generally to indicia of amplification (e.g., a reaction progress parameter) that are measured in time units (e.g., minutes). Time-dependent indicia of amplification are commonly used for monitoring progress in isothermal polynucleotide amplification reactions that are not characterized by distinct “cycles.” All of Time, TArc and OTArc are examples of time-dependent indicia of amplification.

As used herein, the phrase “as a function of” describes the relationship between a dependent variable (i.e., a variable that depends on one or more other variables) and an independent variable (i.e., a variable that may have its value freely chosen without considering the values of any other variables), wherein each input value for the independent variable relates to exactly one output value for the dependent variable. Conventional notation for an equation that relates a y-value (i.e., the dependent variable) “as a function of” an x-value (i.e., the independent variable) is y=f(x).

As used herein, a “computer” is an electronic device capable of receiving and processing input information using software instructions to generate an output. The computer may be a standalone device (e.g., a personal computer), or may be an integrated component of an instrument (e.g., a nucleic acid analyzer that amplifies a target nucleic acid and monitors synthesis of amplification products as a function of reaction cycle number or time). Particularly embraced by the term is an embedded processor resident within an analyzer instrument, and harboring embedded software instructions (sometimes referred to a “firmware”).

As used herein, “optimizing” or “fitting” an equation refers to a process, as commonly practiced in mathematical modeling or curve fitting procedures, for obtaining numerical values for coefficients in an equation to yield an expression that “fits” or approximates experimental measurements. Typically, an optimized equation will define a best-fit curve.

As used herein, the terms “optimized equation,” and “fitted equation” are alternative references to an equation containing fixed numerical values for coefficients as the result of an optimizing procedure. “Fitted” curves (e.g., the products of curve fitting procedures) result from optimizing an equation.

As used herein, a “system” is an arrangement of parts or components organized to cooperate with one another. For example, a system may include an instrument that detects nucleic acids in a sequence-specific manner, and a computer programmed with software to analyze results, where the computer and the instrument are in communication with each other.

As used herein, “apparatus” generally refers to the collection of equipment (e.g., tools, instruments, etc.) needed for a particular purpose or function.

As used herein, an “instrument” is a tool, device, or implement for performing a task. In some embodiments, an instrument is a device contained within a single housing or situated on common support structure (e.g., a single chassis).

By “kit” is meant a packaged combination of materials, typically intended for use in conjunction with each other. Kits in accordance with the present disclosure may include instructions or other information in a “tangible” form (e.g., printed information, electronically recorded on a computer-readable medium, or otherwise recorded on a machine-readable medium such as a bar code for storing numerical values).

As used herein, the term “comprising” and its cognates are used in their inclusive sense; that is, equivalent to the term “including” and its corresponding cognates.

By “consisting essentially of” is meant that additional component(s), composition(s) or method step(s) that do not materially change the basic and novel characteristics of the present invention may be included in the present invention. Any component(s), composition(s), or method step(s) that have a material effect on the basic and novel characteristics of the present invention would fall outside of this term.

Unless defined otherwise all technical and scientific terms used herein have the same meaning as commonly understood to one of ordinary skill in the art to which this invention belongs.

### DETAILED DESCRIPTION OF CERTAIN EMBODIMENTS

Specificity of analyte detection using real-time nucleic acid amplification techniques generally relies on matching primers and probes to complementary target sequences that are to be detected. Reduced specificity can result when attempting to distinguish base sequences that differ from each other only very slightly. Indeed, detection of single nucleotide differences (e.g., “single nucleotide polymorphisms” or “SNPs”) commonly relies on the use of probes that exactly complement the sequences to be detected. Non-specific detection of closely matched sequences (e.g., a wild-type sequence and a SNP differing by a single nucleotide) can be problematic for real-time detection algorithms that rely on run curves exceeding a fluorescence threshold (sometimes “cutoff” herein) value to demonstrate the presence of a target nucleic acid. Stated differently, undesired signals due to cross-hybridization of a labeled probe with non-target amplification product can undesirably exceed a threshold value used for gauging the presence of a target nucleic acid. The present technique overcomes this problem by using a mathematical transformation of run curve data to enhance specific target detection.

### Preferred Polynucleotide Amplification Methods

Examples of in vitro polynucleotide amplification methods useful in connection with the present technique include, but are not limited to: the Polymerase Chain Reaction (PCR), Transcription Mediated Amplification (TMA), Single-Primer Nucleic Acid Amplification, Nucleic Acid Sequence-Based Amplification (NASBA), Strand Displacement Amplification (SDA), Self-Sustained Sequence Replication (3SR), DNA Ligase Chain Reaction (LCR) and amplification methods using self-replicating polynucleotide molecules and replication enzymes such as MDV-1 RNA and Q-heta enzyme. Methods for carrying out these various amplification techniques can be found in U.S. Pat. No. 4,965,188, European Patent No. EP 0 460 828 B1, U.S. Pat. Nos. 5,399,491, 7,374,885, published European patent application EP 0 525 882, U.S. Pat. No. 5,455,166, Guatelli et al., Proc. Natl. Acad. Sci. USA 87:1874-1878 (1990), International Publication No. WO 89/09835, U.S. Pat. No. 5,472,840 and Lizardi et al., Trends Biotechnol. 9:53-58 (1991). The disclosures of these documents which describe how to perform polynucleotide amplification reactions are hereby incorporated by reference.

### Preferred Systems and Apparatus

The methods disclosed herein are conveniently implemented using a computer or similar processing device (“computer” hereafter). In different preferred embodiments, software or machine-executable instructions can be loaded or otherwise held in a memory component of a freestanding computer, or in a memory component of a computer linked to a device used for monitoring, preferably as a function of a reaction progress parameter (e.g., either reaction time or reaction cycle number), the amount of a product undergoing analysis. In a highly preferred embodiment, software for executing the disclosed procedure is held in a memory component of a computer that is linked to, or that is an integral part of a device or apparatus capable of monitoring the amount of an amplicon present in a reaction mixture as a function of reaction cycle number. This includes a processing device component on an electronic circuit board (e.g., embedded software) of an automated nucleic acid analyzer. Generally speaking, the computer is said to be “in communication with” the apparatus that detects and/or quantifies a target nucleic acid when information from the nucleic acid analyzer is transferred from the apparatus to the computer, by any means. Steps instructed by software can include: acquiring a real-time run curve data set representing production of an amplification product in an amplification reaction; calculating a derivative of the real-time run curve data set, or a processed version thereof, including magnitude values of the derivative; comparing the calculated magnitude values of the derivative with a cutoff or threshold value (e.g., a predetermined threshold value); and determining either that a test sample included a target nucleic acid if any calculated magnitude value exceeded the threshold value, or determining that a test sample does not include a target nucleic acid if no calculated magnitude value exceeded the threshold value. In some embodiments, results generated by the computer can be delivered to an output device that displays or records a result of a calculation or comparison. Exemplary output devices include a video monitor and a printer. In some embodiments, the output device is a recording device that produces a “non-transient” record (e.g., a “tangible” record). The non-transient record may be printed on paper, or stored electronically (such as on a computer hard drive or flash drive, magnetic tape or other computer-readable media, etc.).

In some embodiments, the computer can be in communication with, either by wired or wireless means, a fluorometer that detects fluorescent signals, where the fluorometer is arranged or configured to monitor fluorescent signals generated in one or more reaction vessels contained within a temperature-controlled incubator. The incubator can be a temperature-controlled block (e.g., a metal block configured for receiving and containing one or more tubes, or even a multi-well plate), or a chamber that exposes one or more reaction vessels to controlled temperature conditions.

In some embodiments, either or both of a controller system for controlling a real-time amplification device and/or the detection system of the real-time amplification device can be coupled to an appropriately programmed computer that functions to instruct the operation of these instruments in accordance with preprogrammed or user input instructions. The computer preferably also can receive data and information from these instruments, and interpret, manipulate, and report this information to the user.

In some embodiments, the computer also can include appropriate software for receiving user instructions, either in the form of user input into a set of parameter fields, or in the form of preprogrammed instructions (e.g., preprogrammed for a variety of different specific operations). The software then converts these instructions to appropriate language for instructing the operation of the real-time amplification controller to carry out the desired operation. Preferably, the computer also is capable of receiving data from one or more sensors or detectors included within the system, and interpreting the data in accordance with the programming. The system preferably includes software that correlates a feature of a growth curve representing the quantity of amplified copies of the nucleic acid of interest as a function of time, as detected by the detector, to the number of copies of the nucleic acid of interest present in a test sample.

Preferably, when the computer used for executing the disclosed technique is an integral component of an apparatus for performing and analyzing real-time nucleic acid amplification reactions, the apparatus preferably comprises a temperature-controlled incubator, a detection device for collecting signals (e.g., a fluorometer), and an analyzing device (e.g., a computer or processor) for analyzing signals. The apparatus optionally can further include an output device for displaying data obtained or generated. The analyzing device may be connected to the temperature-controlled incubator through an input device known in the art, and/or connected to an output device known in the art for data display. In one embodiment, the temperature-controlled incubator is capable of temperature cycling, and may be configured as a block for receiving one or more tubes, or reaction receptacles (e.g., multi-tube units).

Generally speaking, the various components of an apparatus for performing the real-time nucleic acid amplification useful in connection with the disclosed methods will be conventional components that will be familiar to those having an ordinary level of skill in the art. The temperature-controlled incubator used to perform and analyze real-time nucleic acid amplification may be of a conventional design which can hold a plurality of reaction tubes, or reaction samples in a temperature-controlled block in standard amplification reaction tubes or in wells of a multiwell plate. In one aspect, the detection system is suitable for detecting optical signals from one or more fluorescent labels. The output of the detection system (e.g., signals corresponding to those generated during the amplification reaction) can be fed to the computer for data storage and manipulation. In one embodiment, the system detects multiple different types of optical signals, such as multiple different types of fluorescent labels and has the capabilities of a microplate fluorescence reader. The detection system is preferably a multiplexed fluorimeter containing an excitation light source, which may be a visible light laser or an ultraviolet lamp or a halogen lamp, a multiplexer device for distributing the excitation light to the individual reaction tubes and for receiving fluorescent light from the reaction tubes, a filtering means for separating the fluorescence light from the excitation light by their wavelengths, and a detection means for measuring the fluorescence light intensity. Preferably, the detection system of the temperature-controlled incubator provides a broad detection range that allows flexibility of fluorophore choice, high sensitivity and excellent signal-to-noise ratio. Optical signals received by the detection system are generally converted into signals which can be operated on by the computer or processor to provide data which can be viewed by a user on a display of a user device in communication with the computer or processor. The user device may comprise a user interface or may be a conventional commercially available computer system with a keyboard and video monitor. Examples of data which can be displayed by the user device include amplification plots, scatter plots, sample value screens for all the tubes or reaction vessels in the assembly and for all labels used, an optical signal intensity screen (e.g., fluorescent signal intensity screen), final call results, text reports, and the like.

### Illustrative Nucleic Acid Analyzers and Systems

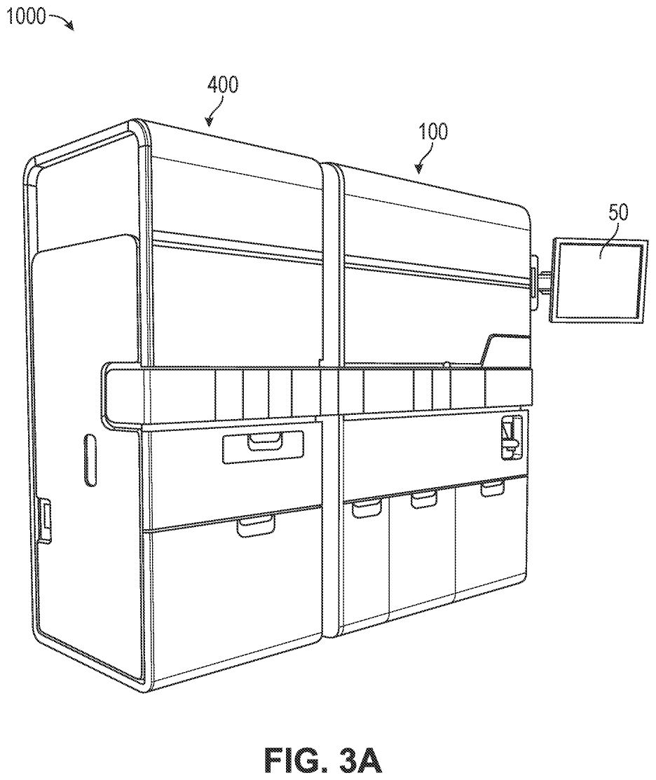

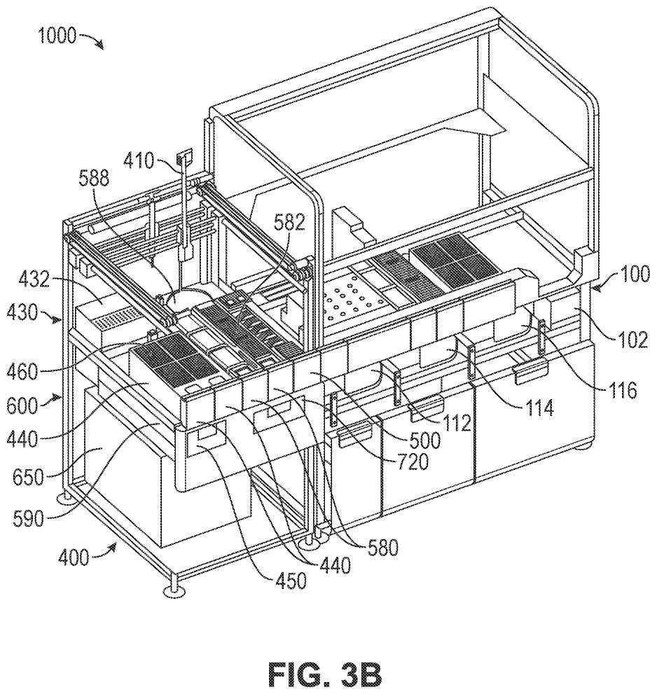

FIGS. 3A and 3B illustrate an exemplary automated analytical system 1000 that may be used to simultaneously analyze a plurality of samples. FIG. 3A is a perspective view of system 1000, while FIG. 3B is view of system 1000 with its canopy removed to show features within. In the discussion below, reference will be made to both FIGS. 3A and 3B. System 1000 is configured to isolate and purify nucleic acid obtained from a plurality of samples introduced into the system, and to amplify and detect targeted nucleic acid contained in any of the samples using differently configured assay reagents. In some embodiments, system 1000 may be a random-access system that allows in vitro diagnostic (IVD) assays and laboratory developed tests (LDTs) to be performed in an interleaved manner. System 1000 may be configured to perform any type of molecular assay. In some embodiments, system 1000 may be configured to perform a plurality of different (e.g., differently configured) molecular assays on a plurality of samples. For example, a plurality of samples may be loaded in system 1000, processed to specifically or non-specifically isolate and purify targeted nucleic acids, subject a first subset of the samples to a first set of conditions for performing a first nucleic acid amplification, and, simultaneously, subject a second subset of the samples to a second set of conditions for performing a second nucleic acid amplification, where the reagents for performing the first and second nucleic acid amplifications are differently configured. In some such embodiments, system 1000 may prompt the user for information using, for example, a graphical user interface (GUI) displayed on a display device 50 (e.g., a computer monitor or a video monitor) of system 1000 (see FIG. 3A) or another display associated with system 1000 (e.g., a remote computer), defining one or more parameters of an assay protocol that can be saved and used later.

In some embodiments, system 1000 may have a modular structure and may be comprised of multiple modules operatively coupled together. However, it should be noted that the modular structure of system 1000 is only exemplary, and in some embodiments, system 1000 may be an integrated system having multiple regions or zones, with each region or zone, for example, performing specific steps of an assay which may be unique to that region. System 1000 includes a first module 100 and a second module 400 operatively coupled together. First module 100 and second module 400 may each be configured to perform one or more steps of an assay. In some embodiments, first and second modules 100, 400 may be separate modules selectively coupled together. That is, first module 100 can be selectively and operatively coupled to second module 400, and first module 100 can be selectively decoupled from second module 400 and coupled to a different second module 400. First and second modules 100, 400 may be coupled together by any method. For example, fasteners (e.g., bolts or screws), clamps, belts, straps, or any combination of fastening/attachment devices may be used to couple these modules together. As explained above, the modular structure of system 1000 is only exemplary, and in some embodiments, system 1000 may be an integral, self-contained structure (with, for example, the first module 100 forming a first region and the second module forming a second region within the integrated structure). It should be noted that in this disclosure, the term “module” is used to refer to a region (zone, location, etc.) of the analytical system. In some embodiments, each such region may be configured to perform specific steps of an assay which may be unique to that region of the system.

In some embodiments, power, data, and/or utility lines or conduits (air, water, vacuum, etc.) may extend between first and second modules 100, 400. In some embodiments, first module 100 may be a system that was previously purchased by a customer, and second module 400 may be a later acquired module that expands the analytical capabilities of the combined system. For example, in one embodiment the first module 100 may be a Panther® system (Hologic Inc.; Marlborough, MA) configured to perform sample processing and isothermal, transcription-based amplification assays (e.g., TMA or NASBA) on samples provided to the system, and module 400 may be a bolt-on that is configured to extend the functionality of the Panther® system by, inter alia, adding thermal cycling capabilities to enable, for example, real-time PCR reactions. An exemplary system 1000 with exemplary first and second modules 100, 400 is the Panther Fusion® system (Hologic Inc., Marlborough, MA), which is described in U.S. Pat. Nos. 9,732,374, 9,465,161, and 9,604,185, and U.S. Patent Publication No. 2016/0032358. Exemplary systems, functions, devices or components, and capabilities of first and second modules 100, 400 are described in the above-referenced publications (and in the publications identified below), and are therefore not described in detail herein for the sake of brevity.

In some embodiments, first module 100 may include multiple vertically stacked decks. As illustrated, first module 100 may be configured to perform one or more steps of a multi-step molecular assay designed to detect at least one analyte (e.g., target nucleic acid). First module 100 may include receptacle-receiving components configured to receive and hold the reaction receptacles and, in some instances, to perform process steps on the contents of the receptacles. Exemplary process steps may include: dispensing sample and/or reagents into reaction receptacles, including, for example, target capture reagents, buffers, oils, primers and/or other amplification oligomers, probes, polymerases, etc.; aspirating material from the reaction receptacles, including, for example, non-immobilized components of a sample or wash solutions; mixing the contents of the reaction receptacles; maintaining and/or altering the temperature of the contents of reaction receptacles; heating or chilling the contents of the reaction receptacles or reagent containers; altering the concentration of one or more components of the contents of the reaction receptacles; separating or isolating constituent components of the contents of the reaction receptacles; detecting a signal, such as electromagnetic radiation (e.g., visible light) from the contents of the reaction receptacles; and/or deactivating nucleic acid or halting on-going reactions.

In some embodiments, first module 100 may include a receptacle drawer or compartment 102 adapted to receive and support a plurality of empty reaction receptacles. Compartment 102 may include a cover or door for accessing and loading the compartment with the reaction receptacles. Compartment 102 may further include a receptacle feeding device for moving the reaction receptacles into a receptacle pick-up position (e.g., a registered or known position) to facilitate removal of the reaction receptacles by a receptacle distributor. First module 100 may further include one or more compartments configured to store containers that hold bulk reagents (i.e., reagent volumes sufficient to perform multiple assays) or are configured to receive and hold waste material. The bulk reagents may include fluids such as, for example, water, buffer solutions, target capture reagents, and nucleic acid amplification and detection reagents. In some embodiments, the bulk reagent container compartments may be configured to maintain the containers at a desired temperature (e.g., at a prescribed storage temperature), and include holding structures that hold and/or agitate the containers to maintain their contents in solution or suspension. An exemplary holding structure for supporting and agitating fluid containers is described in U.S. Pat. No. 9,604,185.

First module 100 may further include a sample bay supporting one or more sample holding racks with sample-containing receptacles. First module 100 may also include one or more fluid transfer devices for transferring fluids, for example, sample fluids, reagents, bulk fluids, waste fluids, etc., to and from reaction receptacles and/or other containers. In some embodiments, the fluid transfer devices may comprise one or more robotic pipettors configured for controlled, automated movement and access to the reaction receptacles, bulk containers holding reagents, and containers holding samples. In some embodiments, the fluid transfer devices may also include fluid dispensers, for example, nozzles, disposed within other devices and connected by suitable fluid conduits to containers, for example, bulk containers holding reagents, and to pumps or other devices for causing fluid movement from the containers to the dispensers. First module 100 may further include a plurality of load stations (e.g., heated load stations) configured to receive sample receptacles and other forms of holders for supporting sample receptacles and reagent containers. An exemplary load station and receptacle holder is described in U.S. Pat. No. 8,309,036.

In some embodiments, first module 100 may include one or more magnetic parking stations and heated incubators 112, 114, 116 configured to heat (and/or maintain) the contents of reaction receptacles at a temperature higher than ambient temperature, and one or more chilling modules configured to cool (and/or maintain) the contents of reaction receptacles at a temperature lower than ambient temperature. Chilling modules may be used to aid in oligo hybridization and/or to cool a receptacle before performing luminescence measurements. In some embodiments, incubator 112 (which may be referred to as a transition incubator) may be set at a temperature of about 43.7° C. and may be used for process steps such as, for example, lysis, target capture, and hybridization. Incubator 114 may be a high temperature incubator which, in some embodiments, may be set at a temperature of about 64° C. and used for process steps such as, for example, lysis, target capture, and hybridization. Incubator 116 (referred to as an amplification incubator) may be set at a temperature of about 42° C., and may be an incubator used for amplification during an assay. Incubator 116 may include real time fluorometers for the detection of fluorescence during amplification. Exemplary temperature ramping stations are described in U.S. Pat. No. 8,192,992, and exemplary incubators are described in U.S. Pat. Nos. 7,964,413 and 8,718,948. First module 100 may include sample-processing devices, such as magnetic wash stations adapted to separate or isolate a target nucleic acid or other analyte (e.g., immobilized on a magnetically-responsive solid support) from the remaining contents of the receptacle.

In some assays, samples are treated to release materials capable of interfering with the detection of an analyte (e.g., a targeted nucleic acid) in a magnetic wash station. To remove these interfering materials, samples may be treated with a target capture reagent that includes a magnetically-responsive solid support for immobilizing the analyte. Suitable solid supports may include paramagnetic particles (0.7-1.05 micron particles, Sera-Mag™ MG-CM (available from Seradyn, Inc., Indianapolis, Indiana). When the solid supports are brought into close proximity to a magnetic force, the solid supports are drawn out of suspension and aggregate adjacent a surface of a sample holding container, thereby isolating any immobilized analyte within the container. Non-immobilized components of the sample may then be aspirated or otherwise separated from immobilized analyte. Exemplary magnetic wash stations are described in U.S. Pat. Nos. 6,605,213 and 9,011,771.

First module 100 may include a detector configured to receive a reaction receptacle and detect a signal (e.g., an optical signal) emitted by the contents of the reaction receptacle. In one implementation, the detector may comprise a luminometer for detecting luminescent signals emitted by the contents of a reaction receptacle and/or a fluorometer for detecting fluorescent emissions from the contents of the reaction receptacle. First module 100 may also include one or more signal detecting devices, such as, for example, fluorometers (e.g., coupled to one or more of incubators 112, 114, 116) configured to detect (e.g., at periodic intervals) signals emitted by the contents of receptacles contained in the incubators while a process, such as nucleic acid amplification, is occurring within the reaction receptacles. Exemplary luminometers and fluorometers are described in U.S. Pat. Nos. 7,396,509 and 8,008,066.

First module 100 may further include a receptacle transfer device, which includes a receptacle distributor configured to move receptacles between various devices of first module 100 (e.g., incubators 112, 114, 116, load stations, magnetic parking stations, wash stations, and chilling modules). These devices may include a receptacle transfer portal (e.g., a port covered by an openable door) through which receptacles may be inserted into or removed from the devices. The receptacle distributor may include a receptacle distribution head configured to move in an X direction along a transport track assembly, rotate in a theta (θ) direction, and move in an R direction, to move receptacles into and out of the devices of first module 100. An exemplary receptacle distributor, exemplary receptacle transfer portal doors, and mechanisms for opening the doors are described in U.S. Pat. No. 8,731,712.

In an exemplary embodiment, second module 400 is configured to perform nucleic acid amplification reactions (such as, for example, PCR), and to measure fluorescence in real-time. System 1000 may include a controller that directs system 1000 to perform the different steps of a desired assay. The controller may accommodate LIS (“laboratory information system”) connectivity and remote user access. In some embodiments, second module 400 houses component modules that enable additional functionalities, such as melt analyses. An example of a melt station that could be adapted for use in the second module is described in U.S. Pat. No. 9,588,069. Other devices may include a computer or controller, a computer hard drive or other memory device, a printer, and an optional uninterruptible power supply.

With reference to FIG. 3B, in some embodiments, second module 400 includes multiple vertically stacked levels (or decks) including devices configured for different functions. These levels include an amplification processing deck 430 and a receptacle processing deck 600. In the illustrated embodiment, receptacle processing deck 600 is positioned below amplification processing deck 430. However, this is not a requirement, and the vertical order of the decks (and their devices) may vary according to the intended use of analytical system 1000. Second module 400 may include devices positioned at different levels. These devices include, among others, a fluid transfer device in the form of one or more robotic pipettor(s) 410 (see FIG. 3B), a thermal cycler 432 with a signal detector, tip compartments 580 configured to store trays of disposable tips for pipettor(s) 410, cap/vial compartments 440 configured to store trays 460 of disposable processing vials and associated caps, a bulk reagent container compartment 500, a bulk reagent container transport, a receptacle distribution system including a receptacle handoff device and a receptacle distribution system including a receptacle distributor (which, in the exemplary embodiment shown, comprises a rotary distributor), receptacle storage units configured to store receptacles and/or multi-receptacle units (MRUs) (that, for example, includes multiple receptacles joined together as a single piece, integral unit), magnetic slots, a waste bin coupled to one or more trash chutes, a centrifuge 588, a reagent pack changer, reagent pack loading stations, and one or more compartments 450 (see FIG. 3B) configured to store accessories, such as, for example, consumables and/or storage trays for post-cap/vial assemblies. Robotic pipettor 410 attaches a disposable fluid transfer tip from a disposable tip tray 582 to a mounting end of its aspirator probe.

Exemplary embodiments of trays 460 for disposable processing vials and caps are disclosed in U.S. Patent Publication No. US 2017/0297027 A1. Several devices and features of system 1000 are described in U.S. Pat. No. 9,732,374 and other references that are identified herein. Therefore, for the sake of brevity, these devices and features are not described in detail herein.

In the illustrated embodiment, robotic pipettor 410 is disposed near the top of second module 400. Below robotic pipettor 410, amplification processing deck 430 includes bulk reagent container compartment 500, centrifuge 588, the top of thermal cycler 432, tip compartments 580, and cap/vial compartments 440. Below amplification processing deck 430, receptacle processing deck 600 includes receptacle handoff device, receptacle distributor, receptacle storage units, magnetic slots, reagent pack changer, and reagent pack loading stations. Magnetic slots and reagent pack loading stations on receptacle processing deck 600 are accessible by robotic pipettor 410 through a gap between the devices of amplification processing deck 430. With reference to FIG. 3B, second module 400 may include a compartment 590 for storing accessories or to accommodate expansion of second module 400 (for example, to add additional reagent compartments for storage of reagents, add analytical capabilities to system 1000, etc.). Trash bin 650 collects and holds used materials, such as used disposable fluid transfer tips. The front surface of second module 400 preferably includes at least one drawer, where each drawer can include a drawer front 720.

The receptacles in the receptacle storage units may include individual receptacles (e.g., a container configured to store a fluid) having an open end and an opposite closed end, or multiple receptacles (e.g., five) coupled together as a unit (MRU). These MRUs may include a manipulating structure that is configured to be engaged by an engagement member (e.g., a hook) of a robotically controlled receptacle distribution system for moving the receptacle between different devices of system 1000. Exemplary receptacles are described in U.S. Pat. Nos. 6,086,827 and 9,732,374. In some embodiments, the receptacle distribution system, including receptacle handoff device and receptacle distributor, is configured to receive a receptacle or an MRU from the receptacle distributor of first module 100 and transfer the receptacle to second module 400, and then move the receptacle into different positions in second module 400.

### Computer Program Products

Included within the scope of the disclosure are software-based products (e.g., tangible embodiments of software for instructing a computer to execute various procedural steps) that can be used for performing the data processing method. These include software instructions stored on a computer or computer-readable media, such as magnetic media, optical media, “flash” memory devices, and computer networks or cloud storage.

The disclosure further embraces a system or an apparatus that amplifies nucleic acids, detects nucleic acid amplification products, and processes results to indicate a quantitative result for target in a test sample. Although the various components of the apparatus preferably function in a cooperative fashion, there is no requirement for the components to be part of an integrated assembly (e.g., on a single chassis). However, in a preferred embodiment, components of the apparatus are connected together. Included within the meaning of “connected” are connections via wired and wireless connections.

Particularly falling within the scope of the disclosure is an apparatus or system that includes a computer linked to a device that amplifies nucleic acids and monitors amplicon synthesis as a function of cycle number or time, where the computer is programmed to execute the algorithmic steps disclosed herein. An exemplary system in accordance with the disclosure will include a temperature-controlled incubator, and a fluorometer capable of monitoring and distinguishing at least two wavelengths of fluorescent emissions. These emissions may be used to indicate target amplicon synthesis, and internal control or internal calibrator amplicon synthesis.

In connection with computer-implemented or software-implemented embodiments of the disclosure, a result can be recorded or stored in a “non-transient” format where it can be accessed for reference at a later time than when the data analysis to be recorded was carried out or performed. For example, a computed result can be recorded in a non-transient format by printing on paper, or by storing on a computer-readable memory device (e.g., a hard drive, flash memory device, file in cloud storage, etc.).

Software instructions in accordance with the disclosure can direct a computer to carry out different steps. For example, the steps may relate to: receiving input signals representing a real-time run curve data set; calculating a derivative of the real-time run curve data set or a processed version thereof (e.g., processed by baseline subtraction and/or curve fitting to smooth the data); comparing magnitudes of the derivative with a threshold value (e.g., a predetermined threshold value); and determining either that a test sample included a target nucleic acid if any calculated magnitude value exceeded the threshold value, or determining that a test sample does not include a target nucleic acid if no calculated magnitude value exceeded the threshold value.

### Curve Fitting Procedures

In accordance with the disclosed method of creating and assessing a real-time run curve, plot, or fitted equation for determining the presence or absence of a target nucleic acid in a test sample, the procedure preferably involves obtaining one or more equations optimized to fit a real-time run curve data set. The data set can comprise signal values (e.g., fluorescent signal values), optionally processed using baseline subtraction and/or curve fitting, as a function of a reaction progress parameter (e.g., reaction cycle number) produced by a nucleic acid analyzer calibrated for determining the amount of a target nucleic acid in a known volume of liquid sample. Optionally, data sets can be normalized using a curve parameter or processed using baseline subtraction. In some embodiments, normalization can involve dividing fluorescence values of the data set by a maximum observed fluorescence value, or some other parameter, of the curve or data set. Processing can be accomplished by applying standard mathematical curve fitting techniques to the data set to result in a fitted equation that defines a curve associated therewith. In some embodiments, the equation used in the curve fitting procedure preferably is a non-linear equation that contains no less than two, more preferably no less than three, and more preferably no less than four coefficients that can be optimized or determined during the curve fitting procedure. Some highly preferred equations have exactly four coefficients, while other highly preferred equations have exactly five coefficients. Optimizing an equation to fit the measured indicia of amplification can easily be accomplished using a commercially available software package, such as the SOLVER program which is available as an EXCEL add-in tool for finding an optimal value for a formula, and equation solving from Microsoft Corporation (Redmond, WA).

Although other equations can be used in the curve fitting procedure, certain preferred methods employed a four-parameter logistic (4-PL) equation having the following form:

$$f ( x ) = A + \frac{B}{( 1 + {( \frac{x}{C} )}^{∧} D )} \qquad ( Eq 1 )$$

In this equation, the dependent variable (y) can represent an observed or processed fluorescent signal as a function of the reaction cycle number (x). The four coefficients in the equation that can be optimized by standard procedures are identified as “a” to “d.” Of course, it is to be understood that success in using the technique of the present disclosure does not require the use of any particular equation.

In some embodiments, calculating the first derivative of a real-time run curve data set involved first fitting the data to an equation to obtain an optimized equation (e.g., an optimized 4-PL equation), and then taking the first derivative of the optimized equation. The derivative of the optimized equation is an equation that can be solved for different input cycle number values (e.g., x-values) to obtain outputs representing calculated derivatives. An example equation expressing the derivative of a fitted 4-PL equation can have the following form.

$${f}^{′} ( x ) = - \frac{BD * {( \frac{x}{C} )}^{D}}{x * {( {( \frac{x}{C} )}^{D} + 1 )}^{2}} \qquad ( Eq 2 )$$

At least three different approaches can be used to determine the derivative of a real-time run curve data set in accordance with the disclosed technique. As described elsewhere herein, data points (x, y) in the real-time run curve data set represent reaction progress parameter values (e.g., time or cycle numbers) and magnitude of production of the amplification product (e.g., fluorescence magnitudes). First, raw or processed run curve data can be used to calculate slope values between adjacent “fitted” data points. Second, an equation optimized to fit the real-time run curve data set in a curve fitting procedure can be solved at selected x-values to calculate corresponding fluorescence values (e.g., y-values). This processed run curve data can be used to calculate slope values between adjacent data points. An example equation that can be used to fit the run curve data is given by Eq 1. Third, an equation for the derivative of an equation optimized to fit the real-time run curve data set can be solved for different x-values (e.g., cycle numbers) to give the corresponding slope value. An example equation for the derivative is given by Eq 2. In each of the three instances, results of the calculations can be expressed, for example, in units of RFU/cycle.

### Alternative Equations for Performing Curve Fitting

Notably, although a 4-PL equation is preferred for fitting or modeling real-time run curves, other mathematical functions can also be used in the procedure with equally good results.

Those having an ordinary level of skill in the art will appreciate that numerous types of equations may be used in the procedures disclosed herein. Examples of symmetric transition functions include, but are not limited to: Sigmoid, Gaussian Cumulative, Lorentzian Cumulative and Cumulative Symmetric Double Sigmoidal. Examples of asymmetric transition functions include, but are not limited to: Logistic Dose Response (LDR), Log Normal Cumulative, Extreme Value Cumulative. Pulse Cumulative, Pulse Cumulative with Power Term, Weibull Cumulative, Asymmetric Sigmoid, Asymmetric Sigmoid Reverse Asymmetry, Cascade Formation, and Cumulative Exponentially Modified Gaussian. Additionally, simple linear and non-linear equations, such as multiple order polynomials, power, exponential and logarithmic functions can be used to model real-time data with subsequent adjustment of the baseline coefficient, as detailed herein. Kinetic functions with baseline coefficients can also be used in the same manner. Exemplary basic kinetic equations containing baseline coefficients include but are not limited to: Half Order Decay and Formation, First Order Decay and Formation, Second Order Decay and Formation, Second Order Decay and Formation (Hyperbolic Forms), and Third Order Decay and Formation, Variable Order Decay and Formation. Exemplary complex kinetic equations containing baseline coefficients include but are not limited to: Simultaneous First and Second Order Decay and Formation, First Order Sequential Formation, Two Component First Order Decay, Two First Order Independent Decay and Formation, Two Second Order Independent Decay and Formation, and First and Second Order Independent Decay and Formation. Exemplary kinetic equilibrium equations containing baseline coefficients include but are not limited to: Simple Equilibrium (Forward and Reverse Rate), Simple Equilibrium (Net Rate and Equilibrium Concentration), Complex Equilibrium A=B+C, and Complex Equilibrium A+B=C+D. Exemplary intermediate kinetic equations containing baseline coefficients include but are not limited to: First Order Intermediate and First Order Intermediate with Equilibrium.

All of the above-listed equation types can be used to carry out the disclosed methods employing fitted curves. This is because success of the procedure depends not on the particular equation used, but on its ability to fit the data optimally. Thus, for example, run curve data can be fitted to an equation (e.g., a 4-PL equation), and derivative analysis can be performed using the fitted equation or curve instead of the raw fluorescence data.

### WORKING EXAMPLES

In the following illustrations, SNP-containing variant nucleic acids represent “target” nucleic acids (desired to be detected), while wild-type nucleic acids represent “non-target” nucleic acids.

Example 1 illustrates how undesired cross-hybridization of SNP-specific probes to amplified wild-type nucleic acid led to false-positive results indicating the presence of a SNP-containing target nucleic acid in test samples. Derivative-based transformation of real-time nucleic acid amplification run curve results, when compared to a fluorescence threshold value, advantageously distinguished true-positive and false-positive results.

### Example 1

### Data Processing Algorithm Resolves Cross-Hybridization Results

Samples of six different model nucleic acid targets (in vitro RNA transcripts and linearized DNA plasmids), each including ribosomal nucleic acid sequences differing at a single nucleotide position, were prepared in a pH buffered sample transport medium (STM) at concentrations ranging from 8.33×10³ to 8.33×10⁵ copies/mL. One of the model targets represented a wild-type sequence, while the remaining five model targets represented SNPs (e.g., sequence variants; model mutant sequences, etc.). Aliquots of each sample were separately incubated in an aqueous solution containing sequence-specific oligonucleotide reagents to facilitate capture of nucleic acid targets onto magnetic microparticles displaying surface oligo-(dT). The magnetic microparticles and bound nucleic acid were separated from the bulk solution by application of a magnetic field. This allowed the supernatant to be removed from the [captured target]:[magnetic microparticle] complex. Magnetic microparticles were washed twice using cycles of resuspension in a wash buffer followed by magnetic separation. Washed microparticles were next incubated in 50 μL of a low ionic strength elution buffer. The magnetic particles were again separated by the application of a magnetic field, and nucleic acid target-containing eluate was recovered.

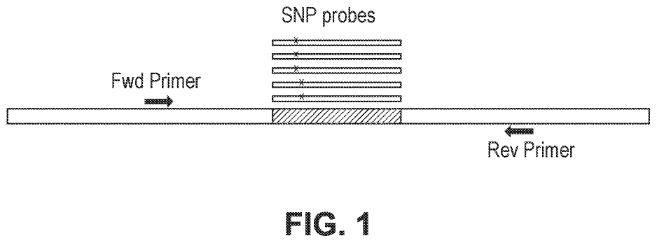

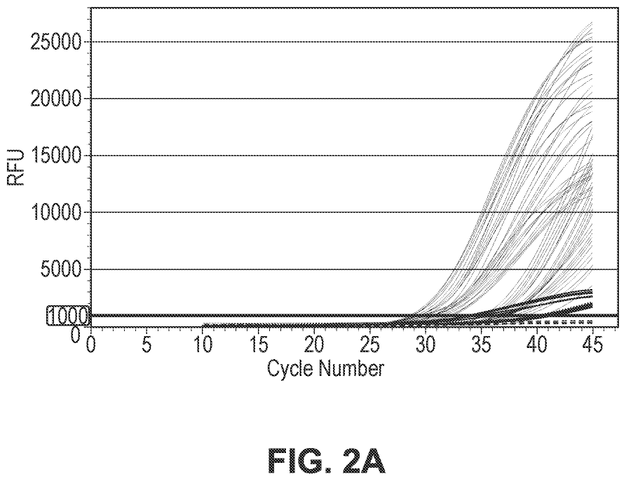

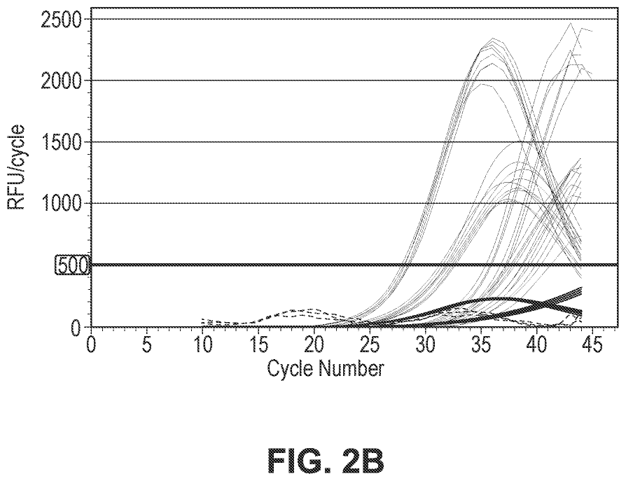

Measured aliquots of each captured and purified model nucleic acid target were distributed to individual reaction vessels and subsequently combined with aliquots of an amplification reagent mixture. The amplification reagent mixture included a reverse transcriptase enzyme, a DNA polymerase enzyme, 4 dNTPs, dUTP, inorganic salts, trehalose, and EDTA. The reagent mixture additionally included a pair of oppositely disposed primers for amplifying the six model nucleic acid targets using primer binding sites common to all of the target nucleic acid templates. Still further, the reagent mixture included six hydrolysis probes of identical length. It is to be noted that use of the probe set was simply a design choice in the procedure. Probes differing in length, but each with a sequence complementary to a different SNP could have been used instead. Each probe harbored a fluorophore and a quencher in energy transfer relationship, and each probe had a base sequence exactly complementary to only one of the model nucleic acid amplification products. The probe complementary to the amplification product representing the wild-type sequence included a CalRed610 fluorophore and a BHQ-2 quencher moiety (Biosearch Technologies, Inc.; Petaluma, CA). Each of the five probes complementary to the SNP variants included a FAM fluorophore (i.e., the same fluorophore) and a BHQ-1 quencher moiety (Biosearch Technologies, Inc.; Petaluma, CA). The general arrangement of primers and probes used for detecting amplification products is schematically illustrated in FIG. 1. Real-time PCR amplification and detection reactions were performed using an automated Panther Fusion® System (Hologic, Inc.; San Diego, CA) to carry out thermal cycling and fluorescence monitoring. The Panther Fusion® System served as an exemplary nucleic acid analyzer in the procedure. Individual amplification reactions were primed using only one of the model nucleic acid targets as a template, but included all six hydrolysis probes. The choice of labels used on the probes meant that fluorescence arising from hydrolysis of SNP-specific probes (used for detecting model variant sequences) could be detected in one fluorescence channel of the instrument, but not substantially detected in a different fluorescence channel that was used for detecting hydrolysis of the wild-type probe. Likewise, fluorescence arising from hydrolysis of the wild-type probe could be detected in one channel of the instrument, but not substantially detected in a different channel that was used for detecting fluorescence arising from hydrolysis of the SNP-specific probes. This permitted all of the probes to be used in the same reaction mixture while still allowing distinction between fluorescence arising from hydrolysis of the SNP-specific probes and fluorescence arising from hydrolysis of the wild-type probe. A negative control that omitted the model nucleic acid target was amplified in replicates of two. All other trials were amplified in replicates of four. Fluorescence readings were collected each cycle of the PCR amplification procedure to yield run curves displaying fluorescence as a function of reaction cycle number. Graphical results presented in FIGS. 2A and 2B illustrate how false-positive detection of SNP-containing target nucleic acids was substantially eliminated using a real-time nucleic acid amplification platform and the disclosed technique involving derivative analysis.

FIG. 2A shows run curves representing fluorescence as a function of reaction cycles (starting from cycle number 10), where fluorescence was produced by FAM-labeled SNP-specific probes in amplification reaction mixtures that included either: (1) 8.33×10⁵ or 8.33×10³ copies/mL of SNP-containing nucleic acid targets (e.g., model variant sequences); (2) wild-type nucleic acid target at a concentration of 8.33×10⁵ or 8.33×10³ copies/mL; or (3) a negative control that omitted both SNP-containing and wild-type nucleic acids altogether. The horizontal fluorescence threshold line shown in the figure had been established to achieve a balance between sensitivity and specificity for detection of SNP-containing nucleic acid targets. Those having an ordinary level of skill in the art will appreciate that such thresholds conventionally are selected to maximize correct results (e.g., true-positives and true-negatives) while minimizing incorrect results (e.g., false-positives and false-negatives). This approach can be used to establish a threshold that can be used for subsequent analyses, where the threshold would be termed a “predetermined threshold” in the subsequent assay. As expected, negative control reactions yielded only very low levels of fluorescence that remained below the horizontal threshold line used for scoring detection of amplification products, and so yielded no Ct values. Ideally, only run curves having a fluorescence signal magnitude that exceeded the horizontal line in FIG. 2A (i.e., this crossing point being the Ct value) would indicate the presence of a SNP-containing nucleic acid target. The SNP-containing nucleic acid targets were clearly amplified and detected in the reaction mixtures, as evidenced by run curves having magnitudes that increased above the horizontal threshold line. Significantly, run curves for reaction mixtures that included wild-type template nucleic acids also were observed to rise above the horizontal threshold between about cycle numbers 34 and 42. Inspection of FIG. 2A reveals that the lower tested concentrations of SNP-containing nucleic acid targets rose above the horizontal threshold at about this same cycle number range. This meant that SNP-containing nucleic acid variants and wild-type nucleic acid targets could not be distinguished from each other by the presence or absence of Ct values (i.e., the point at which a run curve crosses a threshold) in this cycle number range. False-positive errors resulting from cross-hybridization of SNP-specific probes to amplified wild-type nucleic acid targets that are non-complementary at a single nucleotide position could have catastrophic consequences in medical diagnostic applications.

FIG. 2B illustrates a solution to the problem identified in FIG. 2A. The plot in FIG. 2B shows a mathematical derivative of the run curve results presented in FIG. 2A. Generally speaking, the purpose of the derivative analysis was to impose a criterion on run curve shape, in addition to fluorescence magnitude, to establish the presence of SNP-containing nucleic acid target in the test sample undergoing amplification. Although the first derivative of run curves is used here to illustrate the disclosed technique, second, third, or even higher order derivatives may be used in place of the first derivative for the assessment. In the illustrated case, any calculated derivative having a magnitude greater than a threshold value (shown as a horizontal line) indicated the presence of SNP-containing amplification products in the reaction mixture. Any trial yielding calculated derivatives having magnitudes less than the threshold value indicated that SNP-containing amplification products were not included in the reaction mixture.

Example 2 further illustrates how signal arising from undesired cross-hybridization between labeled SNP-specific probe(s) and mismatched wild-type target was discriminated from signal arising from hybridization of SNP-specific probes to amplified variants that included the SNPs. In this procedure, a collection of five detectably labeled probes having sequences specific for five individual SNPs was used in real-time amplification reactions that amplified, one at a time, either corresponding variant targets (“SNP targets”) or a wild-type target that was not exactly complementary to the sequence of any of the five probes. Fluorescent signal arising from hybridization of the labeled SNP-specific probes to mismatched wild-type target amplicon was discriminated using derivative-based analysis of the run curves. All SNP-specific probes harbored the same fluorescent label (e.g., a first fluorescent label). Wild-type target amplicon was detected in control procedures using a wild-type probe that harbored a second fluorescent label (data not shown). In some embodiments, signal arising from the second fluorescent label is not reported to an end-user of the assay. The technique disclosed below illustrates how particular target nucleic acids (e.g., SNP-containing targets, or variant sequences) can be detected and discriminated from a closely matched non-target (e.g., wild-type) sequence that may be susceptible to cross-hybridization of labeled probes. The approach advantageously avoided the need to redesign oligonucleotide primers and probes to be able to make the discrimination. Moreover, the procedure disclosed below allowed for detection of both variant and wild-type sequences using only labeled probes specific for the variant target nucleic acid. In certain preferred embodiments, the procedure is used to detect the presence or absence of a particular type of target nucleic acid (e.g., a variant or mutant target nucleic acid).

### Example 2

### Derivative-Based Analysis Discriminates Closely Related Target Sequences Detected During Real-Time Nucleic Acid Amplification Procedures

Samples of six different in vitro transcripts (IVTs), each IVT corresponding to the same ribosomal nucleic acid sequence but differing at a single nucleotide position, were prepared in a pH buffered sample transport medium (STM) at concentrations ranging from 8.33×10³ to 8.33×10⁶ copies/mL. One of the IVTs represented a wild-type sequence, while the remaining five IVTs represented SNPs or variant sequences. Aliquots of each sample were separately incubated in an aqueous solution containing sequence-specific oligonucleotide reagents to facilitate capture of the IVTs onto magnetic microparticles. The magnetic microparticles and bound nucleic acid were separated from the bulk solution by application of a magnetic field. This allowed the supernatant to be removed from the [captured target]:[magnetic microparticle] complex. Magnetic microparticles were washed twice using cycles of resuspension in a wash buffer followed by magnetic separation. Washed microparticles were next incubated in 50 μL of a low ionic strength elution buffer. The magnetic particles were again separated by the application of a magnetic field, and an IVT-containing eluate was recovered.

Measured aliquots of each captured and purified IVT were distributed to individual reaction vessels and subsequently combined with aliquots of an amplification reagent mixture. The amplification reagent mixture included a reverse transcriptase enzyme, a DNA polymerase enzyme, 4 dNTPs, dUTP, inorganic salts, trehalose, and EDTA. The reagent mixture additionally included a pair of oppositely disposed primers for amplifying the six IVTs using primer binding sites common to all of the target nucleic acid templates. Still further, the reagent mixture included six hydrolysis probes of identical length. Base sequences of probes were the same as the probes in Example 1, but probes in the present example included some optional base modifications. Each probe harbored a fluorophore and a quencher in energy transfer relationship, and each probe had a base sequence exactly complementary to only one of the IVT amplification products. In this Example, probe complementary to the amplification product representing the wild-type sequence included a FAM fluorophore and a BHQ-1 quencher moiety. The five probes complementary to the SNP variants included a CalRed610 fluorophore and a BHQ-2 quencher moiety. Real-time amplification and detection reactions were performed using an automated Panther Fusion® System (Hologic, Inc., San Diego, CA) to carry out thermal cycling and fluorescence monitoring. Emission from the CalRed610 fluorophore was monitored using the ROX detection channel of the Panther Fusion® System, which served as an exemplary nucleic acid analyzer in the procedure. Individual amplification reactions were primed using only one of the IVT target nucleic acid templates, but included all six detectably labeled hydrolysis probes. The choice of labels used on the probes meant that fluorescence arising from hydrolysis of SNP-specific probes (i.e., used for detecting model variant sequences) could be detected in one channel of the instrument (“channel 1”), but not substantially detected in a different channel (“channel 2”) that was used for detecting hydrolysis of the wild-type probe. Likewise, fluorescence arising from hydrolysis of the wild-type probe could be detected in channel 2, but was not substantially detected in channel 1. A negative control that omitted IVT was amplified in replicates of two. All other trials were amplified in replicates of four.

Quantitative results collected using a fluorometer in optical communication with reaction mixtures undergoing amplification were processed to prepare run curves (fluorescence measured as a function of reaction cycle number), calculate derivatives of the run curves, calculate magnitudes of the calculated derivatives, and determine maxima of the calculated magnitudes. These procedures were carried out using programmed computer that was a component of, or in communication with the instrument used for nucleic acid amplification with real-time fluorescence monitoring of amplification product formation. First derivatives were calculated, and maxima of the magnitudes of the first derivatives were determined for the run curves of channel 1. As above, Ct values were determined as the point at which fluorescence run curves crossed a threshold (i.e., a fluorescence threshold or cutoff). If calculated derivative values exceeded a second threshold (i.e., a derivative threshold), then SNP-containing nucleic acid was determined to be present in the amplification reaction. If a derivative did not exceed the second threshold at any point, then the SNP-containing target nucleic acid was not present in the amplification reaction. To simplify presentation of results in tabulated format, maxima of the calculated run curve derivatives were compared to a threshold to determine the presence or absence of variant nucleic acid targets in reaction mixtures.

As demonstrated by the results presented in Table 1, derivative analysis improved specificity of variant sequence detection by distinguishing cycle-dependent signals arising from amplification of variant and non-variant (i.e., wild-type) target nucleic acid templates. Inspection of tabulated results (see column 4) reveals that mean first derivative maxima for variants 1-5 were clearly separated from mean first derivative maxima for trials that included wild-type nucleic acid targets. For example, a threshold of 100 RFU/cycle distinguished these two groups of results. Any first derivative maximum that exceeded the 100 RFU/cycle threshold was judged to indicate detection of variant target nucleic acids. Indeed, trials that included any of variant IVT templates 1-5 met this criterion, and so were judged as positive for the presence of a variant target nucleic acid template containing a SNP. The negative control and trials that included only wild-type templates (i.e., not containing a SNP) uniformly yielded first derivative maxima below the threshold, and so indicated the absence of variant template nucleic acid. This advantageously eliminated false-positive results due to cross-hybridization between SNP-specific probes and the wild-type amplification product. Clearly, if the maximum value of the first derivative did not exceed the threshold required to score positive detection, then no other first derivative value exceeded that threshold. A similar protocol based on determining whether or not the first derivative of a wild-type run curve obtained by monitoring the FAM-labeled probe (detected in channel 2 of the nucleic acid analyzer) exceeded a threshold could have been used for assessing the presence or absence of wild-type template nucleic acid. In this instance, a different predetermined threshold value (i.e., 250 RFU/cycle) was used to detect the presence of amplified wild-type template nucleic acid.

The results presented in Table 1 also showed how Ct values determined using signals produced only by hydrolysis of the SNP-specific probes could be used in combination with derivative results to confirm the presence of either variant or wild-type target nucleic acids. To illustrate, channel 1 fluorescence measured during amplification was evaluated against a set threshold of 250 RFU to determine Ct values. Detecting a Ct value meant that either variant or wild-type nucleic acid was included in the reaction mixture—without distinguishing between the two possibilities. As well, maxima of the first derivative of the fluorescence run curve data were compared to a threshold of 100 RFU/cycle. The presence of a calculated Ct together with a calculated maximum of the run curve first derivative was used to discriminate between variant and wild-type target sequences. More specifically, detection of a Ct value in combination with a first derivative maximum that did not exceed the threshold value meant the reaction included the wild-type nucleic acid target. Detection of a Ct value in combination with a first derivative maximum that exceeded the threshold value meant the reaction included the variant nucleic acid target.

TABLE 1
Detection of Variant and Wild-Type Template
Nucleic Acids using Variant-Specific Probes

| | | | First | | |
|---|---|---|---|---|---|
| | | | Derivative | Either Variant | Variant |
| | | | Maximum | or Wild-Type | Positive (Ct |
| IVT | Concentration | Ct | Mean | Positive | Present and |
| Template | (Copies/mL) | Mean | (Channel 1) | (Ct Present) | Derivative >100) |
| Negative | 0 | X | 7.9 | NEG | NEG |
| Variant 1 | 8.33 × 10³ | 34.7 | 549.9 | POS | POS |
| | 8.33 × 10⁴ | 31.3 | 632.3 | POS | POS |
| | 8.33 × 10⁵ | 27.9 | 668.6 | POS | POS |
| | 8.33 × 10⁶ | 24.7 | 661.5 | POS | POS |
| Variant 2 | 8.33 × 10³ | 38.1 | 130.8 | POS | POS |
| | 8.33 × 10⁴ | 34.7 | 182.6 | POS | POS |
| | 8.33 × 10⁵ | 31.5 | 228.8 | POS | POS |
| | 8.33 × 10⁶ | 28.4 | 206.7 | POS | POS |
| Variant 3 | 8.33 × 10³ | 34.1 | 371.0 | POS | POS |
| | 8.33 × 10⁴ | 30.9 | 447.1 | POS | POS |
| | 8.33 × 10⁵ | 27.5 | 336.4 | POS | POS |
| | 8.33 × 10⁶ | 24.3 | 453.8 | POS | POS |
| Variant 4 | 8.33 × 10³ | 32.9 | 593.9 | POS | POS |
| | 8.33 × 10⁴ | 29.7 | 587.5 | POS | POS |
| | 8.33 × 10⁵ | 26.4 | 660.9 | POS | POS |
| | 8.33 × 10⁶ | 23.0 | 708.8 | POS | POS |
| Variant 5 | 8.33 × 10³ | 33.7 | 508.5 | POS | POS |
| | 8.33 × 10⁴ | 30.3 | 614.0 | POS | POS |
| | 8.33 × 10⁵ | 27.1 | 563.7 | POS | POS |
| | 8.33 × 10⁶ | 23.7 | 563.1 | POS | POS |
| Wild-type | 8.33 × 10³ | 40.3 | 40.9 | POS | NEG |
| | 8.33 × 10⁴ | 36.5 | 62.2 | POS | NEG |
| | 8.33 × 10⁵ | 34.0 | 66.5 | POS | NEG |
| | 8.33 × 10⁶ | 31.1 | 67.2 | POS | NEG |

The results from Example 2 showed how data analysis involving calculation and comparison of magnitudes of a derivative of a real-time run curve increased the specificity of variant sequence detection by distinguishing signals arising from amplification of variant and wild-type target nucleic acid templates. Although trials that included wild-type templates yielded Ct values based on channel 1 fluorescence, signals contributing to establishment of those Ct values were due to cross-hybridization between the wild-type amplification product and SNP probes specific for the variant templates. If Ct values had been used as the only criterion for detecting SNP-containing variant nucleic acid targets, then it would have been erroneously concluded that trials primed with wild-type templates were positive for variant templates. Imposing the additional criterion that required comparison of the first derivative of the run curve to a threshold value advantageously prevented the false-positive assignment. More particularly, the fact that the maximum of the first derivative of run curves primed with wild-type templates fell below the predetermined threshold of 100 RFU/cycle confirmed the templates were wild-type target nucleic acid templates and not variant target nucleic acid templates. In some embodiments, this latter observation was used to indicate the presence of wild-type target nucleic acid templates in the reaction mixture.

Taken together, the results from Example 2 showed how data analysis that involved calculation and comparison of magnitudes of a derivative for a real-time run curve, optionally combined with a threshold-based Ct determination for the run curve, increased the specificity of variant target nucleic acid detection. Amplification reactions yielding a channel 1 (CalRed610) run curve having a first derivative maximum greater than a predetermined threshold value (e.g., 100 RFU/cycle) was sufficient to indicate the presence of a variant target nucleic acid template in the reaction mixture. As briefly addressed above, in some embodiments the presence of amplified wild-type target nucleic acids was indicated by a channel 2 (FAM) first derivative maximum greater than a predetermined threshold. In this instance, a predetermined threshold of 250 RFU/cycle was used to accommodate use of a different fluorescent label. In other embodiments (see Table 1 at columns 3 and 4), detection of a Ct value in the channel 1 (CalRed610) run curve, together with a channel 1 (CalRed610) run curve first derivative maximum that was less than or below the predetermined threshold indicated the absence of variant target nucleic acids in the reaction mixture, and the presence of the wild-type target nucleic acid template in the reaction mixture. By these approaches, both variant and wild-type target nucleic acid templates were detected using labeled probes specific for the variant sequences only.

All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes.

All of the compositions, kits, and methods disclosed and claimed herein can be made and executed without undue experimentation in light of the present disclosure. While the disclosure describes preferred embodiments, it will be apparent to those of skill in the art that variations may be applied without departing from the spirit and scope of the disclosure. All such variations and equivalents apparent to those skilled in the art, whether now existing or later developed, are deemed to be within the spirit and scope of the disclosure.

1. A method of determining whether a test sample suspected of comprising nucleic acid templates for a nucleic acid amplification reaction includes a target nucleic acid, the method comprising the steps of:
(a) acquiring or having acquired a real-time run curve data set comprising signal data representing production of an amplification product in the nucleic acid amplification reaction as a function of a reaction progress parameter,
wherein the nucleic acid amplification reaction uses any of the target nucleic acid and a non-target nucleic acid that may have been present in the test sample as templates to produce the amplification product;
(b) calculating or having calculated a first derivative of the real-time run curve data set, including magnitude values of the first derivative;
(c) comparing or having compared the calculated magnitude values of the first derivative with a first threshold value; and
(d) determining or having determined either that
the test sample includes the target nucleic acid if any calculated magnitude value of the first derivative met or exceeded the first threshold value, or
the test sample does not include the target nucleic acid if no calculated magnitude value of the first derivative met or exceeded the first threshold value.

2. The method of claim 1, wherein the real-time run curve data set comprises fluorescence magnitude readings, and wherein the method further comprises:
comparing the real-time run curve data set with a fluorescence threshold value and determining that at least one data point in the real-time run curve data set has a fluorescence magnitude that exceeds the fluorescence threshold value, and
determining that the test sample comprises the non-target nucleic acid if it is determined in step (d) that the test sample does not include the target nucleic acid.

3. The method of claim 1, wherein the target nucleic acid and the non-target nucleic acid differ from each other at only a single nucleotide position.

4. The method of claim 3, wherein the test sample includes the target nucleic acid, and wherein the method further comprises a step of quantifying or having quantified the target nucleic acid present in the test sample.

5. The method of claim 4, wherein the step of quantifying or having quantified comprises first determining a maximum value of the first derivative from step (b), and then using the maximum value of the first derivative together with the reaction progress parameter as an indicator of the amount of the target nucleic acid present in the test sample.

6. The method of claim 3, further comprising a step of preparing a non-transient record of the result from step (d).

7. The method of claim 6, wherein the non-transient record comprises printing on paper, or recording on computer-readable storage media.

8. The method of claim 5, further comprising a step of preparing a non-transient record of the result from the step of quantifying or having quantified.

9. The method of claim 8, wherein the non-transient record comprises printing on paper, or recording on computer-readable storage media.

10. The method of claim 3, wherein the reaction progress parameter of step (a) is measured in cycle numbers, wherein the nucleic acid amplification reaction comprises a PCR reaction, and wherein the first threshold value is a predetermined threshold value.

11. The method of claim 3, wherein the reaction progress parameter of step (a) is either a measure of reaction time or a measure of reaction cycle number.

12. The method of claim 11, wherein step (a) comprises performing the nucleic acid amplification reaction and monitoring synthesis of amplification products as the nucleic acid amplification reaction is occurring.

13. The method of claim 3, wherein step (a) comprises receiving the real-time run curve data set as a computer-readable data file.

14. The method of claim 1, wherein before step (b) the real-time run curve data set acquired in step (a) is processed using at least one of (i) baseline subtraction, (ii) curve normalization using a curve parameter, and (iii) curve fitting.

15. The method of claim 14, wherein the real-time run curve data set acquired in step (a) is processed using curve fitting, and wherein the curve fitting comprises optimizing coefficients of an equation to result in an optimized equation.

16. The method of claim 1, wherein the real-time run curve data set comprises fluorescent readings measured as a function of the reaction progress parameter, and wherein the reaction progress parameter is measured in reaction cycles.

17. The method of claim 1, wherein step (b) comprises calculating with a computer, and wherein step (c) comprises comparing with the computer.

18. The method of claim 1, wherein the nucleic acid amplification reaction is performed using an automated nucleic acid analyzer configured to isolate nucleic acid from the test sample and then perform the nucleic acid amplification reaction using the isolated nucleic acid, and wherein step (b) comprises calculating with a computer in communication with the automated nucleic acid analyzer, and wherein step (c) comprises comparing with the computer.

19. The method of claim 1, wherein the first threshold value is a numerical constant.

20. The method of claim 1, wherein the target nucleic acid is a target nucleic acid isolated from a human pathogen.

21. The method of claim 20, wherein the human pathogen is either a bacterial pathogen or a viral pathogen.

22. A computer programmed with software instructions to determine whether a target nucleic acid is included in a test sample, the software instructions, when executed by the computer, cause the computer to:
(a) receive a real-time run curve data set comprising signal data that indicates amplification of the target nucleic acid and a non-target nucleic acid in a nucleic acid amplification reaction as a function of a reaction progress parameter;
(b) calculate a first derivative of the real-time run curve data set or a processed version thereof, including magnitude values of the first derivative;
(c) compare the calculated magnitude values of the first derivative with a first threshold value; and
(d) determine either that
the test sample included the target nucleic acid if any calculated magnitude value of the first derivative met or exceeded the first threshold value, or
the test sample did not include the target nucleic acid if no calculated magnitude value of the first derivative met or exceeded the first threshold value.

23. The computer of claim 22, wherein the software instructions, when executed by the computer, further cause the computer to
compare the real-time run curve data set with a fluorescence threshold value to determine whether any signal data of the real-time run curve data set has a magnitude that meets or exceeds the fluorescence threshold value, and
determine that the test sample includes the non-target nucleic acid that differs from the target nucleic acid if the computer determines that the magnitude meets or exceeds the fluorescence threshold value and if the computer determines in (d) that the test sample did not include the target nucleic acid.

24. The computer of claim 22, wherein the signal data in (a) that indicates amplification of the target nucleic acid and the non-target nucleic acid comprises fluorescent signal data.

25. The computer of claim 24, wherein the software instructions, when executed by the computer, further cause the computer to (e) generate a non-transient record of the result from (d).

26. The computer of claim 22, wherein the software instructions, when executed by the computer, cause the computer to prepare the processed version of the real-time run curve data set, and then (b) calculate the first derivative of the processed version the real-time run curve data set.

27. The computer of claim 22, wherein the software instructions, when executed by the computer, further cause the computer to prepare the processed version of the real-time run curve data set by performing at least one of (i) baseline subtraction, (ii) curve normalization using a curve parameter, and (iii) curve-fitting; and wherein (b) comprises calculate the first derivative of the processed version of the real-time run curve data set.

28. The computer of claim 23, wherein the first threshold value in (c) is a numerical constant.

29. The computer of claim 25, wherein the non-transient record in (e) is stored electronically on a computer hard drive.

30. The computer of claim 22, wherein the computer is in communication with a thermal cycling device equipped with a fluorometer.

31. A system that determines whether a target nucleic acid is included in a test sample, the system comprising:
a nucleic acid analyzer configured to amplify the target nucleic acid and a non-target nucleic acid in a nucleic acid amplification reaction,
wherein the nucleic acid amplification reaction uses any of the target nucleic acid and the non-target nucleic acid that may have been present in the test sample as templates to produce an amplification product, and
wherein the nucleic acid analyzer monitors synthesis of the amplification product in the nucleic acid amplification reaction as a function of a reaction progress parameter, whereby there is produced a real-time run curve data set comprising signal data as a function of the reaction progress parameter; and
a computer in communication with the nucleic acid analyzer, the computer being programmed with a set of software instructions causing the computer to
(a) calculate a first derivative of the real-time run curve data set or a processed version thereof, including magnitude values of the first derivative;
(b) compare the calculated magnitude values of the first derivative with a first threshold value;
(c) determine either that
the test sample included the target nucleic acid if any calculated magnitude value of the first derivative met or exceeded the first threshold value, or
the test sample did not include the target nucleic acid if no calculated magnitude value of the first derivative met or exceeded the first threshold value; and
(d) generate a non-transient record of the result from (c).

32. The system of claim 31, wherein the set of software instructions further cause the computer to
compare the real-time run curve data set with a fluorescence threshold value to determine whether any signal data of the real-time run curve data set has a magnitude that meets or exceeds the fluorescence threshold value, and
determine that the test sample includes the non-target nucleic acid that differs from the target nucleic acid if the computer determines that the magnitude meets or exceeds the fluorescence threshold value and if the computer determines in (d) that the test sample did not include the target nucleic acid.

33. The system of claim 31, wherein the set of software instructions further cause the computer to calculate a quantity of the target nucleic acid included in the test sample.

34. The system of claim 33,
wherein the set of software instructions further cause the computer to prepare the processed version of the real-time run curve data set by performing at least one of (i) baseline subtraction, (ii) curve normalization using a curve parameter, and (iii) curve-fitting, and
wherein (a) comprises calculate the first derivative of the processed version of the real-time run curve data set.

35. The system of claim 31, wherein the computer is a stand-alone computer that is not physically joined to the nucleic acid analyzer.

36. The system of claim 31, wherein the computer is in communication with an electronic storage device, and wherein the electronic storage device stores an electronic form of the non-transient record generated by the computer.

37. The system of claim 31, wherein the computer is in communication with a printer that produces the non-transient record.

38. The system of claim 31, wherein the nucleic acid analyzer comprises a fluorometer that detects fluorescent signals produced in the nucleic acid amplification reaction, and wherein the fluorometer is used to monitor synthesis of the amplification product in the nucleic acid amplification reaction.
